(19) United States
(12) Patent Application Publication
(10) Pub. No.: US 2003/0199590 A1
(43) Pub. Date: Oct. 23, 2003

(54) Prostaglandin analogues for promotion of hair growth

(76) Inventors: Gerald D Cagle, Fort worth, TX (US); Michael V.W. Bergamini, Fort worth, TX (US)

Correspondence Address: ALCON RESEARCH, LTD., R&D COUNSEL, Q-148, 6201 SOUTH FREEWAY, FORT WORTH, TX, 76134-2099, US

(21) Appl. No.: 10/275,543

(22) PCT Filed: Jul. 25, 2002
(86) PCT No.: PCT/US02/23584

(51) Int. Cl. A61K 31/557

(52) U.S. Cl. 514/573; 514/651

(57) ABSTRACT

Methods and compositions for the promotion of hair growth in mammals, comprising PGF_{2α} analogues are disclosed.

### FIELD OF THE INVENTION

This invention relates to the use of certain prostaglandin analogues to promote the growth, thickness, and pigmentation of hair in mammals, including man. The invention also relates to topical compositions for such use. More specifically, the invention relates to the use of certain FP prostaglandin analogues for the promotion of hair growth.

### BACKGROUND OF THE INVENTION

Although hair loss has plagued mankind for centuries, its cause is still not completely understood and no adequate cure has yet been found. Two of the more recent commercial products for alopecia or male pattern baldness are minoxidil (Rogaine®) and finasteride (Propecia®). The active compounds of both these products were initially developed for different therapies: minoxidil for hypertension, and finasteride for benign prostatic hypertrophy. See U.S. Pat. Nos. 4,139,619 and 4,968,812 directed to the use of minoxidil, and U.S. Pat. No. 5,981,543 for finasteride. To the extent U.S. Pat. Nos. 4,139,619 and 4,968,812 disclose topical formulations for hair growth promotion, those disclosures are by this reference incorporated herein.

More recently, it has been discovered that prostaglandin analogues, originally developed as therapy for glaucoma, may also promote hair growth. See U.S. Pat. Nos. 6,262,105 B1 and U.S. patent application Ser. No. 09/774,555 (U.S. Patent Application Publication No. US 2002/0037914A1), the entire contents of both of which are by this reference incorporated herein.

The claimed invention in U.S. Pat. No. 6,262,105 (the '105 patent) was based in part on the observation that in some instances glaucoma patients receiving the prostaglandin analogue, latanoprost, experienced increased eyelash growth. The patent broadly discloses the use of prostaglandins and prostaglandin analogues for enhancing hair growth, generically disclosing countless prostaglandin derivatives and analogues, as well as over a hundred specific compounds. Characterized as preferred among the prostaglandin analogues disclosed in the '105 patent were those of the A, F, and E types; and particularly preferred was 13,14-dihydro-15-dehydro-17-phenyl-18,19,20-trinor-PGF_{2α} and its carboxylic acid esters. The compounds useful in the methods and compositions of the present invention, however, are neither specifically disclosed nor suggested in the '105 patent.

U.S. patent application Ser. No. 09/774,555 (the '555 application) discloses 5,6-13,14-tetrahydro PGF_{2α} analogues (also characterized as 13,14-dihydro PGF_{1α} analogues) for treating hair loss. Once again, the compounds useful in the methods and compositions of the present invention are neither disclosed or suggested in the '555 application, nor are they encompassed within the scope of that application's broadest claims.

Cloprostenol and fluprostenol, both known compounds, are synthetic analogues of PGF_{2α} a naturally-occurring F-series prostaglandin (PG). Structures for PGF_{2α} (I), cloprostenol (II), and fluprostenol (III), are shown below:

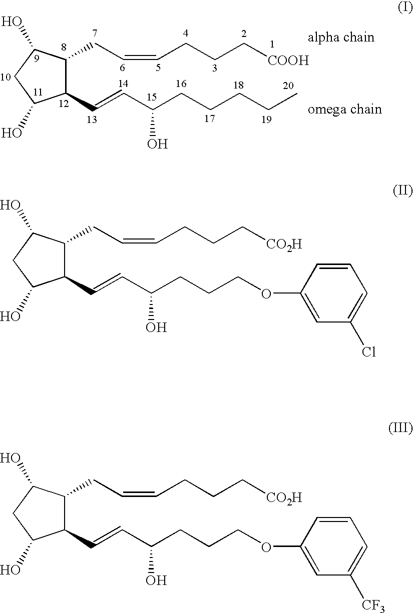

The chemical name for cloprostenol is 16-(3-chlorophenoxy)-17,18,19,20-tetranor PGF_{2α}. Monograph No. 2461 (page 407) of The Merck Index, 12th Edition (1996) is incorporated herein by reference to the extent that it describes the preparation and known pharmacological profiles of cloprostenol. Fluprostenol has the chemical name 16-(3-trifluoromethylphenoxy)-17,18,19,20-tetranor PGF_{2α}. Monograph No.4231 (page 711) of The Merck Index, 12th Edition (1996) is incorporated herein by reference to the extent that it describes the preparation and known pharmacological profiles of fluprostenol. Cloprostenol and fluprostenol are 16-aryloxy PGs differing from the natural product PGF_{2α} by the substitution of a substituted phenoxy moiety for the last 4 carbons of the lower (omega) chain of the compound.

The use of salts and esters of cloprostenol and fluprostenol and various analogues thereof for the treatment of glaucoma and ocular hypertension are described in U.S. Pat. Nos. 5,510,383 and 5,889,052, the entire contents of which are by this reference incorporated herein.

It has now been discovered that particular PGF_{2α} analogues are surprisingly effective agents for the promotion of hair growth when topically applied to mammals including man.

### SUMMARY OF THE INVENTION

The present invention relates to methods and compositions for the promotion of hair growth comprising certain PGF_{2α} analogues and their pharmaceutically acceptable salts, esters, and amides. Most preferred in such compositions and methods are potent and selective FP agonists selected from the group consisting of 9-deoxy, 11-oxa, 13-oxa, and 15-fluoro analogues of PGF_{2α}, and the analogues described in U.S. Pat. Nos. 5,510,383 and 5,889,052, previously incorporated by reference. Encompassed within the scope of the invention are various pharmaceutically acceptable carriers suitable for topical administration.

### DETAILED DESCRIPTION OF THE INVENTION

The compounds useful in the present invention are described in U.S. Pat. Nos. 5,510,383, 5,889,052, 5,698,733, 6,025,392, and 6,232,344; and U.S. patent application Ser. Nos. 09/284,432 and 10/100,399; the entire contents of each of the foregoing being incorporated herein by this reference. Among those compounds are those selected from the group consisting of the compounds of formula:

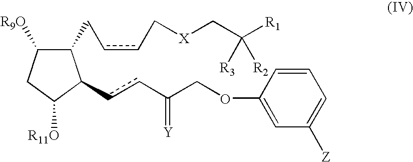

wherein:

R₁═OR, where R═H; C₁-C₁₂ straight-chain or branched alkyl; C₁-C₁₂ straight-chain or branched acyl; C₃-C₈ cycloalkyl; or a cationic salt moiety; or R═NR⁴R⁵, where R⁴ and R⁵ are the same or different and are H; C₁-C₁₂ straight-chain or branched alkyl; C₁-C₁₂ straight-chain or branched acyl; or C₃-C₈ cycloalkyl;

R₂, R₃═H, or C₁-C₅ straight-chain or branched alkyl; or R₂ and R₃ taken together may represent O;

X═O, S, or CH₂;

————represents any combination of a single bond, or a cis or trans double bond for the alpha (upper) chain; and a single bond or trans double bond for the omega (lower) chain;

R₉═H, C₁-C₁₀ straight-chain or branched alkyl, or C₁-C₁₀ straight-chain or to branched acyl;

R₁₁═H, C₁-C₁₀ straight-chain or branched alkyl, or C₁-C₁₀ straight-chain or branched acyl;

Y═O; or H and OR₁₅ in either configuration wherein R₁₅═H, C₁-C₁₀ straight-chain or branched alkyl, or C₁-C₁₀ straight-chain or branched acyl; and

Z═Cl or CF₃;

and compounds 1-80 identified in Table 1 below.

TABLE 1
FP Prostaglandin Analogues

| | | FP binding | FP functional |
|---|---|---|---|
| | MOL STRUCTURE | IC₅₀ (nM) | EC₅₀ nM (%) |
| 1 | 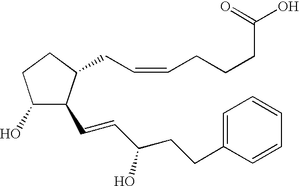 | 96 | 19.6(87.6) |
| 2 | 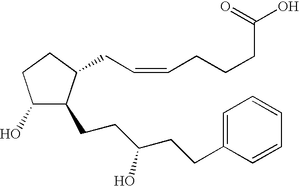 | 310 | 76.6(60) |
| 3 | 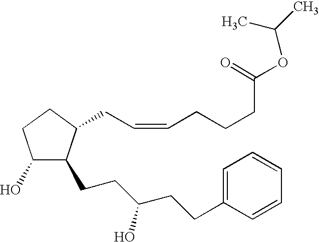 | | |
| 4 | 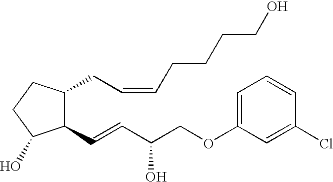 | | |
| 5 | 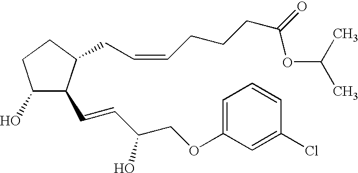 | | |
| 6 | 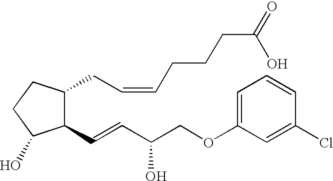 | 10 | 2.6(100) |
| 7 | 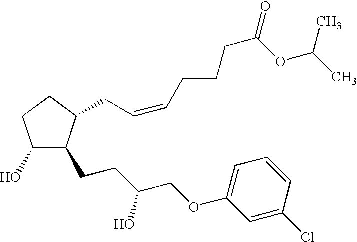 | | |
| 8 | 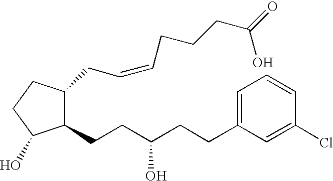 | 47 | 6.6(84) |
| 9 | 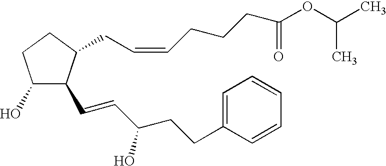 | | |
| 10 | 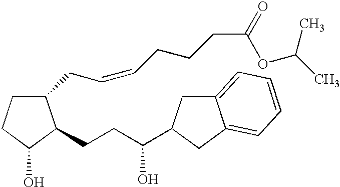 | | |
| 11 | 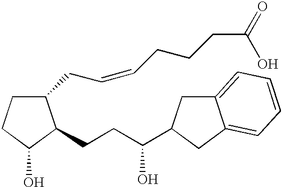 | 1,900 | 108(60) |
| 12 | 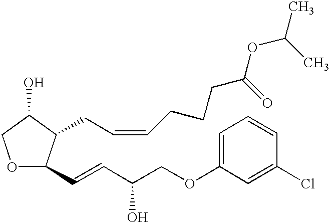 | | |
| 13 | 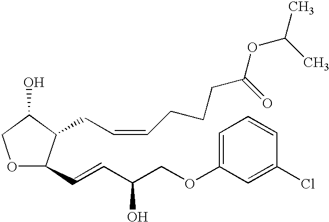 | | |
| 14 | 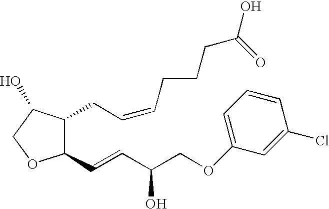 | 7,300 | 194(67) |
| 15 | 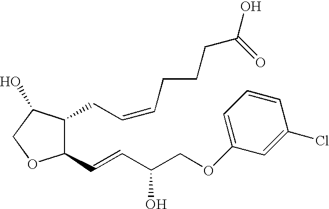 | 890 | 58(88) |
| 16 | 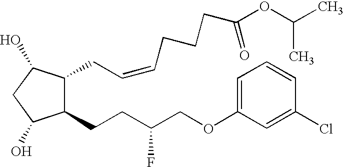 | | |
| 17 | 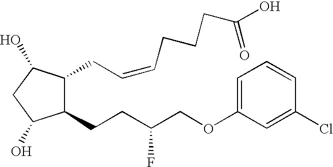 | 26 | 9(64) |
| 18 | 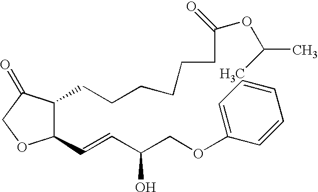 | | |
| 19 | 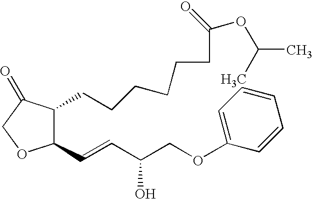 | | |
| 20 | 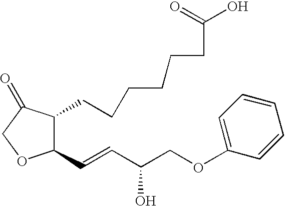 | 9,700 | 10,000(67) |
| 21 | 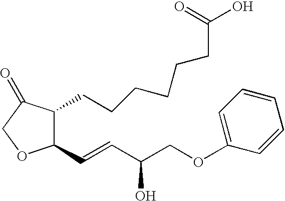 | 78,000 | >10,000 |
| 22 | 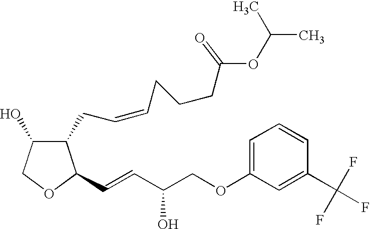 | | |
| 23 | 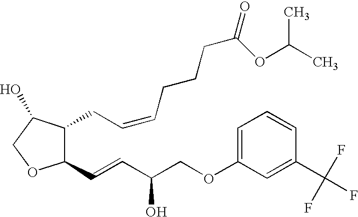 | | |
| 24 | 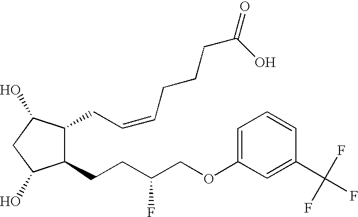 | 230 | 12.5(85) |
| 25 | 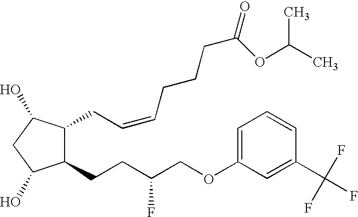 | | |
| 26 | 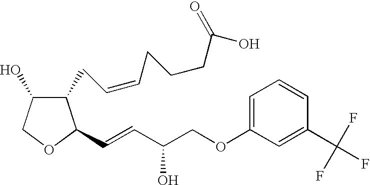 | 1,800 | 187(74) |
| 27 | 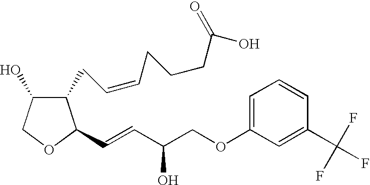 | 15,000 | 889(43) |
| 28 | 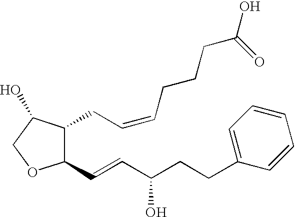 | 1,900 | 80(100) |
| 29 | 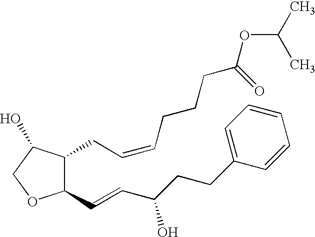 | | |
| 30 | 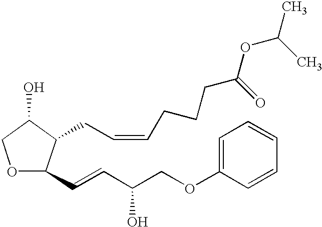 | | |
| 31 | 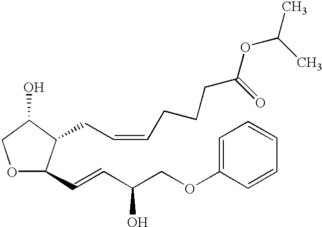 | | |
| 32 | 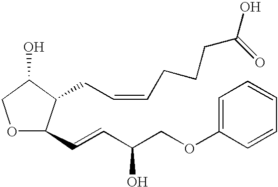 | 573 | 48.6(100) |
| 33 | 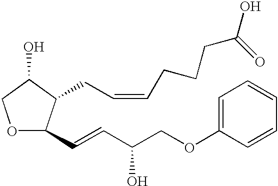 | 640 | 293(67) |
| 34 | 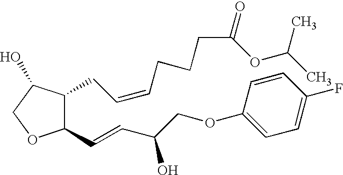 | | |
| 35 | 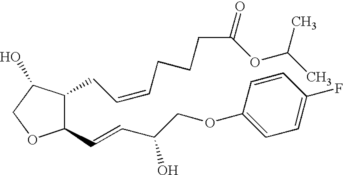 | | |
| 36 | 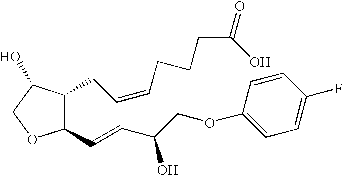 | 4,600 | 1240(75) |
| 37 | 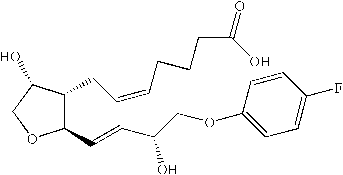 | 1,400 | 437(78) |
| 38 | 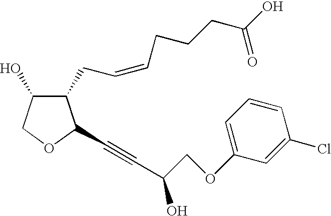 | 36,000 | >10,000 |
| 39 | 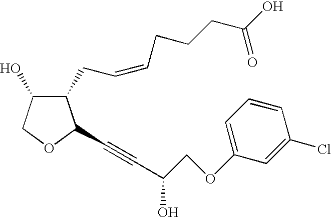 | 5,100 | 509(100) |
| 40 | 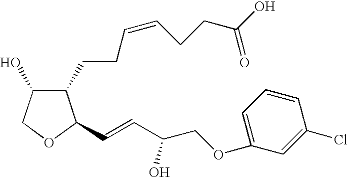 | 430 | 23(82) |
| 41 | 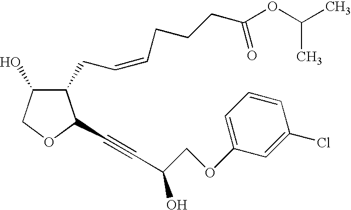 | | |
| 42 | 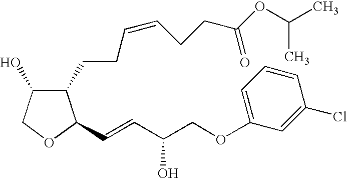 | | |
| 43 | 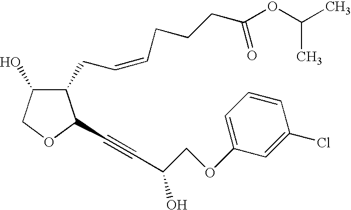 | | |
| 44 | 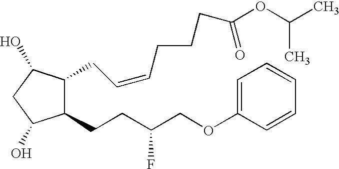 | | |
| 45 | 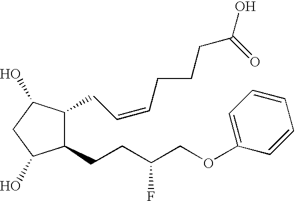 | 52 | 9.5(79) |
| 46 | 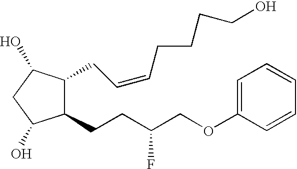 | | |
| 47 | 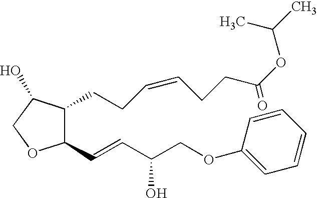 | | |
| 48 | 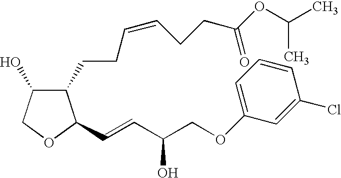 | | |
| 49 | 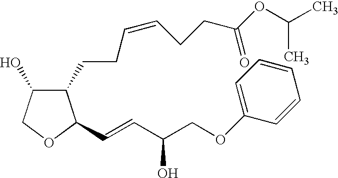 | | |
| 50 | 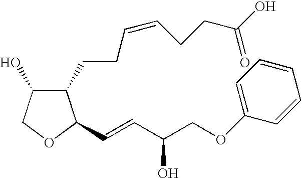 | 5,100 | 252(59) |
| 51 | 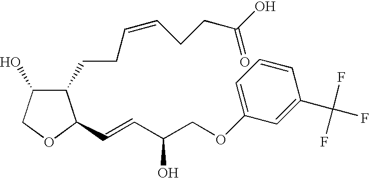 | 9,900 | 781(61) |
| 52 | 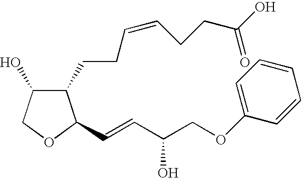 | 330 | 57(83) |
| 53 | 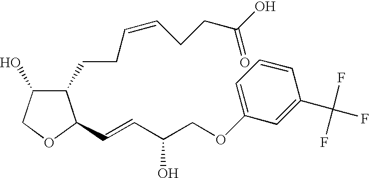 | 350 | 96(92) |
| 54 | 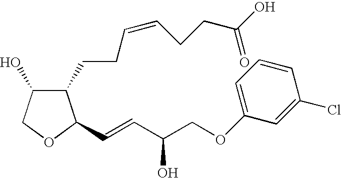 | 818 | 249(71) |
| 55 | 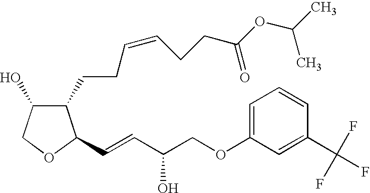 | | |
| 56 | 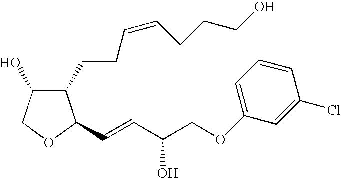 | | |
| 57 | 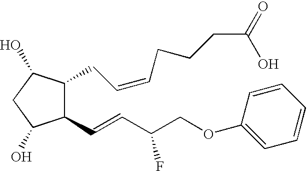 | 267 | 3.8(104) |
| 58 | 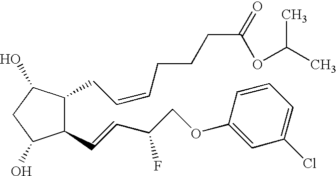 | | |
| 59 | 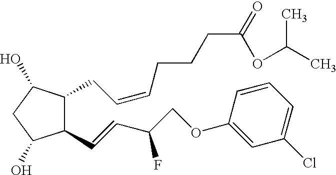 | | |
| 60 | 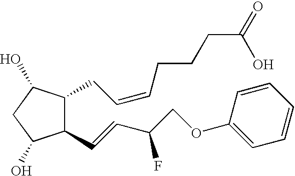 | 1120 | 96(98) |
| 61 | 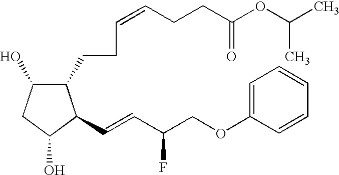 | | |
| 62 | 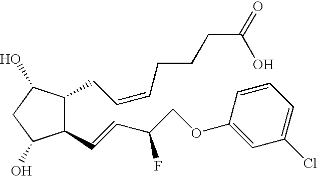 | 182 | 81(76) |
| 63 | 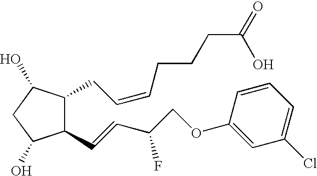 | 238 | 2.2(84) |
| 64 | 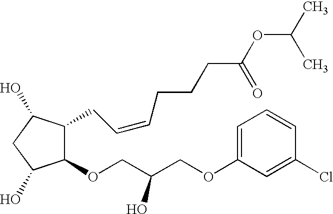 | | |
| 65 | 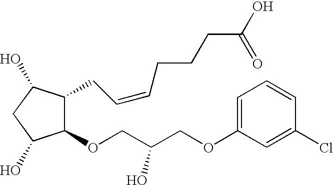 | 540 | 57.8(91) |
| 66 | 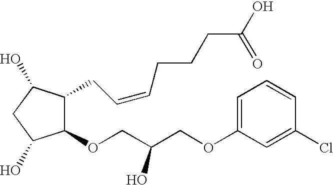 | 4340 | 503(56) |
| 67 | 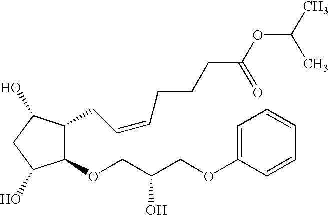 | | |
| 68 | 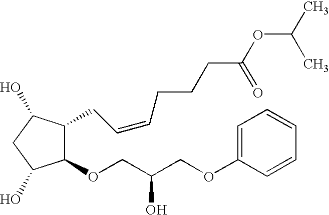 | | |
| 69 | 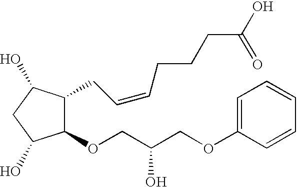 | 277 | 56(57) |
| 70 | 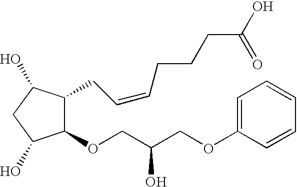 | 1070 | 166(57) |
| 71 | 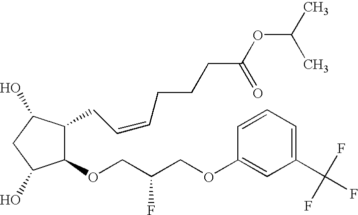 | | |
| 72 | 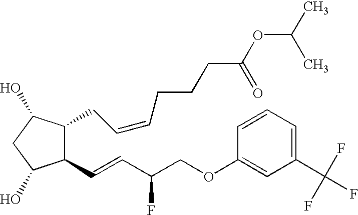 | | |
| 73 | 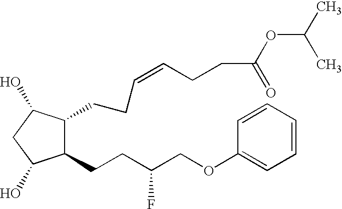 | | |
| 74 | 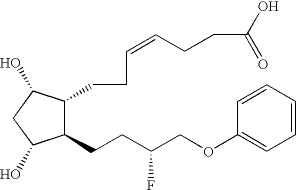 | 690 | 8.8(67) |
| 75 | 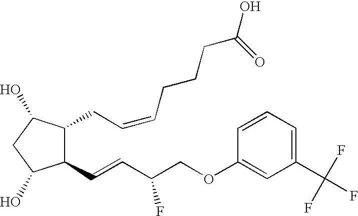 | 160 | 11.1(83) |
| 76 | 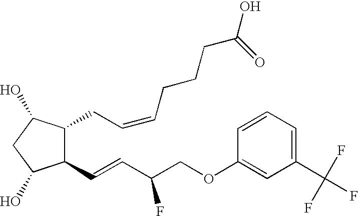 | 4,200 | 131(44.5) |
| 77 | 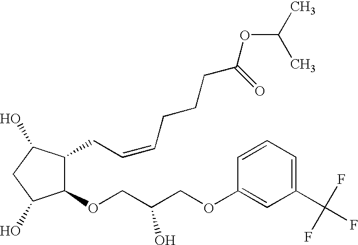 | | |

Preferred among the compounds of formula IV for use in the present invention are those having the structure of formula V:

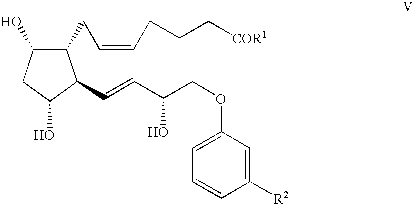

wherein:

R¹═OR or NR³R⁴, where

R═H, a cationic salt moiety, a pharmaceutically acceptable amine moiety, or

C₁-C₁₂ alkyl, cycloalkyl, or aryl;

R³ and R⁴═same or different ═H, alkyl, cycloalkyl, aryl, or OR⁵, with the proviso that R³ and R⁴ cannot both ═OR⁵, where

R⁵═H, alkyl, acyl, cycloalkyl, or aryl; and

R²═Cl or CF₃.

Particularly preferred among such compounds are esters of cloprostenol and fluprostenol, which correspond to formula V wherein:

R¹═OR, where

R═C₁-C₁₂ alkyl; and

R²═Cl or CF_{3.}

Most preferred are the isopropyl esters of cloprostenol and fluprostenol in enantiomercially pure form, which correspond to compounds having the absolute stereochemical structure of formula V wherein:

R¹═OR, where R is isopropyl; and

R²═Cl or CF₃.

Also preferred are the 15-keto analogues of cloprostenol and fluprostenol.

The generic name for the isopropyl ester of the preferred enantiomer of fluprostenol is travoprost. Travoprost, as well as fluprostenol and cloprostenol, and their 15-keto analogues are commercially available from Cayman Chemical Company, Ann Arbor, Mich.

As used herein:

The term “acyl” represents a group that is linked by a carbon atom that has a double bond to an oxygen atom and single bond to another carbon atom.

The term “alkenyl” includes straight or branched chain hydrocarbon groups having 1 to 15 carbon atoms with at least one carbon-carbon double bond. The chain hydrogens may be substituted with other groups, such as halogen. Preferred straight or branched alkenyl groups include, allyl, 1-butenyl, 1-methyl-2-propenyl and 4-pentenyl.

The term “alkoxy” represents an alkyl group attached through an oxygen linkage.

The term “alkyl” includes straight or branched chain, saturated or unsaturated aliphatic hydrocarbon groups having 1 to 15 carbon atoms. The alkyl groups may be substituted with other groups, such as halogen, hydroxyl or alkoxy. Preferred straight or branched alkyl groups include lower alkyl groups such as methyl, ethyl, propyl, isopropyl, butyl and t-butyl. Also included, however, are alkenyl and alkynyl groups.

The term “alkynyl” includes straight or branched chain hydrocarbon groups having 1 to 15 carbon atoms with at least one carbon-carbon triple bond. The chain hydrogens may be substituted with other groups, such as halogen. Preferred straight or branched alkynyl groups include, 2-propynyl, 2-butynyl, 3-butynyl, 1-methyl-2-propynyl and 2-pentynyl.

The term “aryl” refers to carbon-based rings which are aromatic. The rings may be isolated, such as phenyl, or fused, such as naphthyl. The ring hydrogens may be substituted with other groups, such as lower alkyl, or halogen. As used herein, “aryl” includes heteroaryl groups.

The term “cationic salt moiety” includes alkali and alkaline earth metal salts as well as ammonium salts.

The term “cycloalkyl” includes straight or branched chain, saturated or unsaturated aliphatic hydrocarbon groups which connect to form one or more rings, which can be fused or isolated. The rings may be substituted with other groups, such as halogen, hydroxyl or lower alkyl. Preferred cycloalkyl groups include cyclopropyl, cyclobutyl, cylopentyl and cyclohexyl.

The term “heteroaryl” refers to aromatic hydrocarbon rings which contain at least one heteroatom such as O, S, or N in the ring. Heteroaryl rings may be isolated, with 5 to 6 ring atoms, or fused, with 8 to 10 atoms. The heteroaryl ring(s) hydrogens or heteroatoms with open valency may be substituted with other groups, such as lower alkyl or halogen. Examples of heteroaryl groups include imidazole, pyridine, indole, quinoline, furan, thiophene, pyrrole, tetrahydroquinoline, dihydrobenzofuran, and dihydrobenzindole.

The term “lower alkyl” represents alkyl groups containing one to six carbons (C₁-C₆).

Preferred among the compounds of Table 1 are those which are potent and selective FP receptor agonists; specifically, those for which the free acid form exhibits an FP binding IC₅₀ (based upon a travoprost 0.004% standard) of less than 1000 nM and/or greater than 50% agonistic activity (as reflected in Table 1—travoprost acid representing the standard full agonist (100%); and most preferably those with IC₅₀s less than 500 and/or agonistic activity greater than 75%. Most preferred among the compounds of Table 1 are compounds 9, 18, 25, 42, 71 and 77, all of which are isopropyl esters.

The preferred isopropyl esters isopropyl esters will preferably be in enantiomerically pure form, which corresponds to compounds having the absolute stereochemical structure of PGF_{2α}(I).

Representative compounds of formula V are presented in Table 2, which includes corresponding FP receptor binding and agonistic activity data.

TABLE 2

| | | | EC₅₀, |
|---|---|---|---|
| | | | nM (% |
| Com- | | Kᵢ, | re- |
| pound | Structure | nM | sponse) |
| 81 | 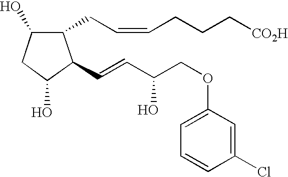 | 91 | 1.4 (100%) |
| 82 | 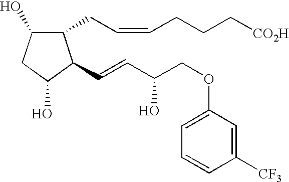 | 35 | 3.8 (96%) |
| 83 | 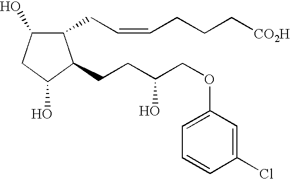 | 50 | 4 (100%) |
| 84 | 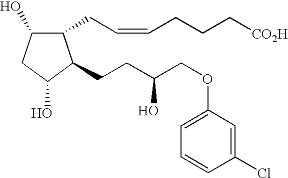 | 130 | 210 (76%) |
| 85 | 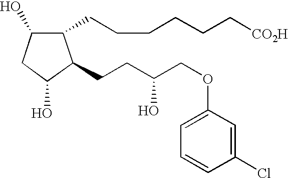 | 220 | 23 (100%) |
| 86 | 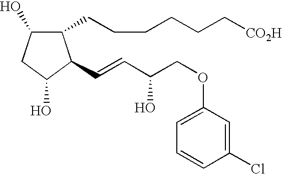 | 93 | 4.92 (100%) |
| 87 | 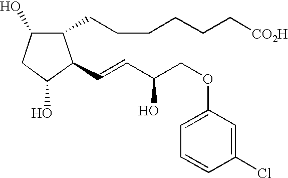 | 730 | 38 (88%) |
| 88 | 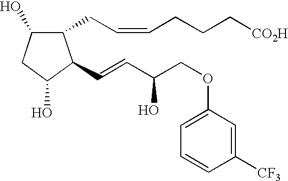 | 150 | 11.6 (99%) |
| 89 | 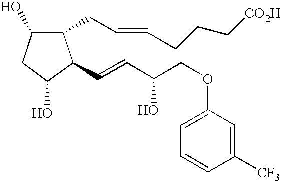 | 260 | 41 (96%) |

Topical formulations containing the prostaglandin analogues of the present invention for the treatment of glaucoma and ocular hypertension as well as methods of synthesizing and formulating the same are disclosed in U.S. Pat. Nos. 5,510,383; 5,698,733; 5,889,052; 6,025,392; and 6,232,344; U.S. patent application Ser. Nos. 09/284,432 and 10/100,399, the entire contents of which were previously incorporated by reference. Storage-stable formulations and packaging systems for the compounds of the present invention are also described in U.S. Pat. Nos. 5,631,287; 6,011,062; and 6,235,781, the entire contents of each of which are by this reference incorporated herein.

The invention is also related to dermatological compositions for topical treatment for the stimulation of hair growth which comprise an effective hair growth stimulating amount of one or more prostaglandin analogues as defined above and a dermatologically compatible carrier. Effective amounts of the active analogues will vary analogues on the derivative employed, frequency of application and desired result, but will generally range from about 0.0000001 to about 50% by weight of the dermatological composition; preferably from about 0.00001 to about 5% by weight; and most preferably from about 0.0001 to about 0.1% by weight. Representative compositions may thus comprise from about 0.001 to about 50 μg of the analogues in about 1 to about 100 μg of total dermatological composition, more preferably from about 0.01 to about 5 μg in about 10 to about 50 μg of the composition.

In forming compositions for topical administration, the compounds of the present invention are generally formulated as between about 0.00003 to about 3 percent by weight (wt %) solutions in water at a pH between 4.5 to 8.0. The compounds are preferably formulated as between about 0.0003 to about 0.3 wt % and, most preferably, between about 0.003 and about 0.03 wt %. While the precise regimen is left to the discretion of the clinician, it is recommended that the resulting solution be topically applied by spray, roll-on or dropper and massaged into the affected area, for example the scalp, once a day.

Other methods of administration include “brushing in,” especially as in the conventional application of mascara to eye lashes. Those skilled in the art will appreciate that the previously described amounts and concentrations of the PGF_{2α} analogues of the present invention may be added to conventional mascara formulations. Examples of mascara formulations may be found in U.S. Pat. No. 6,274,131, the contents of which are by this reference incorporated herein.

Other ingredients which may be desirable to use in the dermatalogical preparations of the present invention include preservatives, co-solvents and viscosity building agents.

Antimicrobial Preservatives:

Dermatological products are typically packaged in multidose form, which generally require the addition of preservatives to prevent microbial contamination during use. Suitable preservatives include: benzalkonium chloride,thimerosal, chlorobutanol, methyl paraben, propyl paraben, phenylethyl alcohol, edetate disodium, sorbic acid, ONAMER M®, or other agents known to those skilled in the art. Such preservatives are typically employed at a concentration between about 0.001% and about 1.0% by weight.

Co-Solvents:

Prostaglandins, and particularly ester derivatives, typically have limited solubility in water and therefore may require a surfactant or other appropriate co-solvent in the composition. Such co-solvents include: Polysorbate 20, 60 and 80; Pluronic F-68, F-84 and P-103; Tyloxapol®; Cremophor® EL; sodium dodecyl sulfate; glycerol; PEG 400; propylene glycol; cyclodextrins; or other agents known to those skilled in the art. Such co-solvents are typically employed at a concentration between about 0.01% and about 2% by weight.

Viscosity Agents:

Viscosity greater than that of simple aqueous solutions may be desirable to increase ocular absorption of the active compound, to decrease variability in dispensing the formulations, to decrease physical separation of components of a suspension or emulsion of formulation and/or otherwise to improve the ophthalmic formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose or other agents known to those skilled in the art. Such agents are typically employed at a concentration between about 0.01% and about 2% by weight.

Included within the scope of the present invention are the individual enantiomers of the title compounds, as well as their racemic and non-racemic mixtures. The individual enantiomers can be enantioselectively synthesized from the appropriate enantiomerically pure or enriched starting material by means such as those described below. Alternatively, they may be enantioselectively synthesized from racemic/non-racemic or achiral starting materials. (Asymmetric Synthesis by J. D. Morrison and J. W. Scott, Ed., Academic Press Publishers: New York, 1983-1985 (five volumes published over a three year span with chapters contributed by about two dozen authors) and Principles of Asymmetric Synthesis by R. E. Gawley and J. Aube, Ed., Elsevier Publishers: Amsterdam, 1996). They may also be isolated from racemic and non-racemic mixtures by a number of known methods, e.g. by purification of a sample by chiral HPLC (A Practical Guide to Chiral Separations by HPLC, G. Subramanian, Ed., VCH Publishers: New York, 1994; Chiral Separations by HPLC, A. M. Krstulovic, Ed., Ellis Horwood Ltd. Publishers, 1989), or by enantioselective hydrolysis of a carboxylic acid ester sample by an enzyme (Ohno, M.; Otsuka, M. Organic Reactions, volume 37, page 1 (1989)). Those skilled in the art will appreciate that racemic and non-racemic mixtures may be obtained by several means, including without limitation, nonenantioselective synthesis, partial resolution or even mixing samples having different enantiomeric ratios. Also included within the scope of the present invention are the individual isomers substantially free of their respective enantiomers.

### EXAMPLE 1

Data for Tables 1 and 2 were generated using the following methodologies.

FP receptor binding assay: The bovine corpus luteum has been shown to express high-affinity [³H]PGF_{2α} binding sites, in addition to [³H]PGE₂ binding, which appear to have pharmacological characteristics of FP receptors. Washed total particulate bovine corpus luteum membranes (20 mg/ml final) were incubated with [³H]PGF_{2α} (0.9-1.5 nM) in Krebs buffer (pH 7.4) for 2 h at 23° C. in a total volume of 500 ml. Non-specific binding was defined with 1-10 μM unlabeled PGF_{2α} or fluprostenol. The assays were terminated by vacuum filtration (using Whatman GF/B glass fiber filter previously soaked in 0.3% polyethyleneimine) and the data analyzed by a non-linear, iterative, curve-fitting computer program.

FP receptor-mediated phosphoinositide turnover assay: [³H]Inositol phosphates ([³H]-IPs) produced by agonist-mediated activation of phospholipase C in Swiss 3T3 cells expressing FP receptors were quantified as follows. Confluent 3T3 cells were exposed to 1.0-1.5 μCi [³H]-myo-inositol (18.3 Ci/mmol) in 0.5 ml DMEM for 24-30 hours at 37° C. Then cells were rinsed once with DMEM/F-12 containing 10 mM LiCl, and the agonist stimulation experiment was performed in 0.5 ml of the same medium to facilitate accumulation of [³H]-IPs. Cells were exposed to the agonist or solvent for 60 min at 37° C. (triplicate determinations), followed by aspiration of the medium and immediate addition of 1 ml of ice-cold 0.1 M formic acid. The plates were kept cold and then frozen. Samples frozen up to one week were thawed prior to chromatographic separation of radiolabeled components. The cell lysates (0.9 ml) were loaded on columns packed with approximately 1 ml AG 1-X8 anion exchange resin. The elution procedure consisted of a wash with 10 ml of H₂O, then 8 ml of 50 mM ammonium formate, and finally 4 ml of 1.2 M ammonium formate with 0.1 M formic acid, which was collected in a scintillation vial. To this eluate was added 15 ml of scintillation fluid and the total [³H]-IPs determined by scintillation counting on a beta-counter. Data were analyzed by the sigmoidal fit function of the Origin Scientific Graphics software (Microcal Software, Northampton, Mass.) to determine agonist potency (EC₅₀ value) and efficacy, relative to the standard cloprostenol.

### EXAMPLE 2

The following Formulations 1-8 are representative pharmaceutical compositions of the invention for topical use in promoting hair growth. Each of Formulations 1 through 8 may be formulated in accordance with procedures known to those skilled in the art.

| | |
|---|---|
| Ingredient | Amount (wt %) |

FORMULATION 1

| | |
|---|---|
| Travoprost | 0.004 |
| Dextran 70 | 0.1 |
| Hydroxypropyl methylcellulose | 0.3 |
| Sodium chloride | 0.77 |
| Potassium chloride | 0.12 |
| Disodium EDTA | 0.05 |
| Benzalkonium chloride | 0.01 |
| HCl and/or NaOH | pH 7.2-7.5 |
| Purified water | q.s. to 100% |

FORMULATION 2

| | |
|---|---|
| Travoprost | 0.004 |
| Monobasic sodium phosphate | 0.05 |
| Dibasic sodium phosphate | 0.15 |
| (anhydrous) | |
| Sodium chloride | O.75 |
| Disodium EDTA | 0.01 |
| Benzalkonium chloride | 0.02 |
| Polysorbate 80 | 0.15 |
| HCl and/or NaOH | pH 7.3-7.4 |
| Purified water | q.s. to 100% |

FORMULATION 3

| | |
|---|---|
| Travoprost | 0.004 |
| Dextran 70 | 0.1 |
| Hydroxypropyl methylcellulose | 0.5 |
| Monobasic sodium phosphate | 0.05 |
| Dibasic sodium phosphate | 0.15 |
| (anhydrous) | |
| Sodium chloride | 0.75 |
| Disodium EDTA | 0.05 |
| Benzalkonium chloride | 0.01 |
| NaOH and/or HCl | pH 7.3-7.4 |
| Purified water | q.s. to 100% |

FORMULATION 4

| | |
|---|---|
| Travoprost | 0.004 |
| Monobasic sodium phosphate | 0.05 |
| Dibasic sodium phosphate | 0.15 |
| (anhydrous) | |
| Sodium chloride | 0.75 |
| Disodium EDTA | 0.05 |
| Benzalkonium chloride | 0.01 |
| HCl and/or NaOH | pH 7.3-7.4 |
| Purified water | q.s. to 100% |

FORMULATION 5

| | |
|---|---|
| Compound 42 | 0.004 |
| Dextran 70 | 0.1 |
| Hydroxypropyl methylcellulose | 0.3 |
| Sodium chloride | 0.77 |
| Potassium chloride | 0.12 |
| Disodium EDTA | 0.05 |
| Benzalkonium chloride | 0.01 |
| HCl and/or NaOH | pH 7.2-7.5 |
| Purified water | q.s. to 100% |

FORMULATION 6

| | |
|---|---|
| Compound 42 | 0.004 |
| Monobasic sodium phosphate | 0.05 |
| Dibasic sodium phosphate | 0.15 |
| (anhydrous) | |
| Sodium chloride | 0.75 |
| Disodium EDTA | 0.01 |
| Benzalkonium chloride | 0.02 |
| Polysorbate 80 | 0.15 |
| HCl and/or NaOH | pH 7.3-7.4 |
| Purified water | q.s. to 100% |

FORMULATION 7

| | |
|---|---|
| Compound 42 | 0.004 |
| Dextran 70 | 0.1 |
| Hydroxypropyl methylcellulose | 0.5 |
| Monobasic sodium phosphate | 0.05 |
| Dibasic sodium phosphate | 0.15 |
| (an hydrous) | |
| Sodium chloride | 0.75 |
| Disodium EDTA | 0.05 |
| Benzalkonium chloride | 0.01 |
| NaOH and/or HCl | pH 7.3-7.4 |
| Purified water | q.s. to 100% |

FORMULATION 8

| | |
|---|---|
| Compound 42 | 0.004 |
| Monobasic sodium phosphate | 0.05 |
| Dibasic sodium phosphate | 0.15 |
| (anhydrous) | |
| Sodium chloride | 0.75 |
| Disodium EDTA | 0.05 |
| Benzalkonium chloride | 0.01 |
| HCl and/or NaOH | pH 7.3-7.4 |
| Purified water | q.s. to 100% |

### EXAMPLE3

Eyelash photographs of each eye were taken at determination of patient eligibility 8AM (baseline) and Months 1.5, 3, 4.5 and 6 in four clinical studies. Additional photographs were taken at Months 9 and 12 in two clinical studies. Eyelash change was classified using the following categories: change in eyelash color, increase in eyelash length, increase in eyelash density and increase in eyelash thickness. Two independent readers evaluated the photographs subjectively for any change from baseline in eyelash characteristics. If the two readers did not agree, the photographs were evaluated by a third reader. In the case when all three readers had a different assessment, a meeting was held to come to a consensus. Eyelash changes from baseline evaluated by photographs were reported and are summarized in the following Table 3.

TABLE 3
Percent of Patients with Eyelash Change

| Treatment | N | % | Total N |
|---|---|---|---|
| Travoprost 0.0015% | 285 | 48.1 | 592 |
| Travoprost 0.004% | 361 | 61.3 | 589 |
| Latanoprost 0.005% | 50 | 25.8 | 194 |
| Total | 835 | 35.4 | 2360 |

The majority of eyelash changes associated with travoprost (0.0015% and 0.004%) includes changes in color, and increases in length, density and/or thickness (Table 4). No clinically significant difference within a treatment group was observed for eyelash color, length, density and/or thickness. A concentration-related change in eyelash color, length, density and/or thickness was observed between patients receiving Travoprost 0.0015% and 0.004%. Patients receiving latanoprost 0.005% experienced similar types of changes in eyelash color, length, density, and/or thickness, but at a greatly reduced frequency compared to that of travoprost.

TABLE 4
Percent of Patients with Eyelash Change by Category in Study

| | Change Reported | | Color Change | | Length Change | | Density Change | | Thickness Change | |
|---|---|---|---|---|---|---|---|---|---|---|
| Treatment | N | % | N | % | N | % | N | % | N | % |
| Travoprost 0.0015% | 285 | 48.1 | 221 | 37.3 | 285 | 48.1 | 264 | 44.6 | 200 | 33.8 |
| N = 592 | | | | | | | | | | |
| Travoprost 0.004% | 361 | 61.3 | 282 | 47.9 | 360 | 61.1 | 343 | 58.2 | 301 | 51.1 |
| N-589 | | | | | | | | | | |
| Latanoprost 0.005% | 50 | 25.8 | 32 | 16.5 | 50 | 25.8 | 43 | 22.2 | 34 | 17.5 |
| N-194 | | | | | | | | | | |

The compounds of the present invention may be formulated and applied as topical creams, ointments, solutions or suspensions, lotions, aerosols, dusting powders and the like. For topical use on the skin and the scalp, the prostaglandin analogues of the present invention can be advantageously formulated using ointments, creams, liniments or patches as a carrier of the active ingredient. Also, these formulations may or may not contain preservatives, depending on the dispenser and nature of use. Such preservatives include those mentioned above, and methyl-, propyl-, or butyl-parahydroxybenzoic acid, betain, chlorhexidine, benzalkonium chloride, and the like. Various matrices for slow release delivery may also be used. Typically, the dose to be applied on the scalp is in the range of about 0.1 ng to about 100 mg per day, more preferably about 1 ng to about 10 mg per day, and most preferably about 10 ng to about 1 mg per day dependingn on the prostaglandin analogue and the formulation. To achieve the daily amount of medication depending on the formulation, the prostaglandin analogue may be administered once or several times daily with or without antioxidants. Those skilled in the art will appreciate that the compounds of the present invention, and especially travoprost or compound 42 may be substituted for the compounds described in examples 2-11 of the '105 patent.

Moreover, the compounds of the present invention may be combined with one or more known agents for the promotion of hair growth. While bound by no theories, the inventors suspect that the mechanism of action by which the compounds of the present invention promote hair growth may be distinct from those of the presently available commercial hair growth products. Specifically, the compounds of the present invention may be combined with: i) minoxidil (Pharmacia) and minoxidil-type compounds; ii) finasteride (Merck) and finasteride-type compounds (dihydrotestosterone (DHT) blockers); and iii) copper-peptides or retinoic acid related compounds. These three types of conventional hair growth products are described at http://www.skinbiology.com/hairregrowth.html (as published Jul. 24, 2001), the contents of which are by this reference incorporated herein. Examples of minoxidil-type compounds include: aminexil (Dercap) (L'Oreal); cromakalim/BRL 34915 (Pharmacia); diazoxide (Hyperstat IV, Proglycem); pinacidil; and its analogue PC-1075. An example of a finasteride-type compound would be the herbal product Saw palmetto (serenoa repens), which acts as a DHT blocker.

The invention has been described by reference to certain preferred embodiments; however, it should be understood that it may be embodied in other specific forms or variations thereof without departing from its spirit or essential characteristics. The embodiments described above are therefore considered to be illustrative in all respects and not restrictive, the scope of the invention being indicated by the appended claims rather than by the foregoing description.

What is claimed is:

1. A method for promoting hair growth in a mammal, comprising the topical application to mammalian skin of an effective amount of a PGF_{2α} analogue selected from the group consisting of: 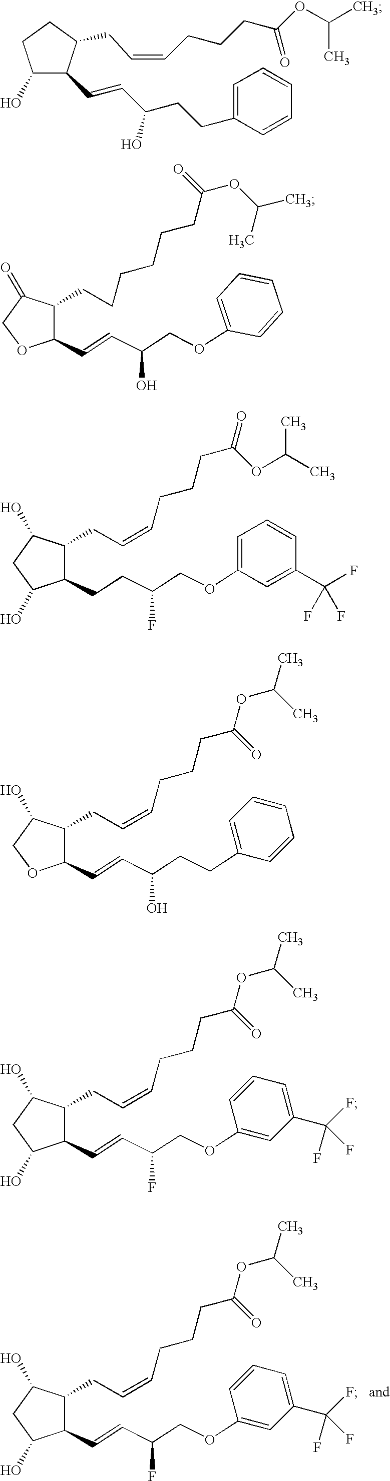
compounds of formula IV: 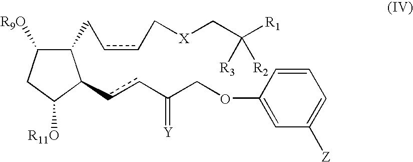
wherein:
R₁═OR, where R═H; C₁-C₁₂ straight-chain or branched alkyl; C₁-C₁₂ straight-chain or branched acyl; C₃-C₈ cycloalkyl; or a cationic salt moiety; or R═NR⁴R⁵, where R⁴ and R⁵ are the same or different and are H; C₁-C₁₂ straight-chain or branched alkyl; C₁-C₁₂ straight-chain or branched acyl; or C₃-C₈ cycloalkyl;
R₂, R₃═H, or C₁-C₅ straight-chain or branched alkyl; or R₂ and R₃ taken together may represent O;
X═O, S, or CH₂;
————represents any combination of a single bond, or a cis or trans double bond for the alpha (upper) chain; and a single bond or trans double bond for the omega (lower) chain;
R₉═H, C₁-C₁₀ straight-chain or branched alkyl, or C₁-C₁₀ straight-chain or branched acyl;
R₁₁═H, C₁-C₁₀ straight-chain or branched alkyl or C₁-C₁₀ straight-chain or branched acyl;
Y═O; or H and OR₁₅ in either configuration wherein R₁₅═H, C₁-C₁₀ straight-chain or branched alkyl, or C₁-C₁₀ straight-chain or branched acyl; and
Z═Cl or CF₃.

2. The method of claim 1, wherein the PGF_{2α} analogue is a compound of formula IV having the structure of formula V: 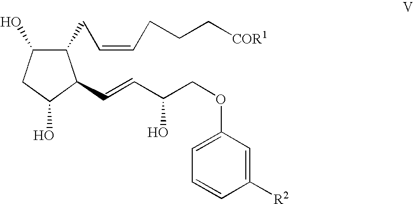
wherein:
R¹═OR or NR³R⁴, where
R═H, a cationic salt moiety, a pharmaceutically acceptable amine moiety, or
C₁-C₁₂ alkyl, cycloalkyl, or aryl;
R³ and R⁴═same or different ═H, alkyl, cycloalkyl, aryl, or OR⁵, with the proviso that R³ and R⁴ cannot both ═OR⁵, where
R⁵═H, alkyl, acyl, cycloalkyl, or aryl; and
R²═Cl or CF₃.

3. The method of claim 2, wherein the compound of formula V is travoprost.

4. The method of claim 1, wherein the PGF_{aα} analogue is applied in a composition comprising from about 0.0000001% to about 50% by weight of said PGF_{aα} analogue.

5. The method of claim 4, wherein the composition comprises from about 0.0001% to about 5% by weight of the PGF_{2α} analogue.

6. The method of claim 5, wherein the composition comprises from about 0.0001% to about 0.1% by weight of the PGF_{2α} analogue.

7. The method of claim 2, wherein the PGF_{2α} analogue is applied in a composition having a concentration from about 0.0001% to about 0.1% by weight of the PGF_{2α} analogue.

8. The method of claim 7, wherein the PGF_{2α} analogue is travoprost.
